(19) United States
(12) Patent Application Publication
(10) Pub. No.: US 2022/0258277 A1
(43) Pub. Date: Aug. 18, 2022

(54) Laser Beam Processing Apparatuses and Correspondent Method Using Multi-beam Interference

(71) Applicant: Joyce Liu, Ithaca, NY (US)

(72) Inventor: Joyce Liu, Ithaca, NY (US)

(21) Appl. No.: 17/175,511

(22) Filed: Feb. 12, 2021

(51) Int. Cl. B23K 26/0622; B23K 26/06; B23K 26/067; B23K 26/21; B23K 26/362; B23K 26/382; B23K 26/18; A61B 18/20

(52) U.S. Cl. CPC B23K 26/0622; B23K 26/0643; B23K 26/0648; B23K 26/0652; A61B 18/20; B23K 26/21; B23K 26/362; B23K 26/382; B23K 26/18; B23K 26/067

(57) ABSTRACT

The invention relates to apparatuses and correspondent method of laser beam processing for various materials with strong light absorption and scattering. The apparatuses can be used for medical no incision laser surgery, long distance underwater or atmosphere light communication, less attenuation light energy delivery in optical turbid media, and so on. This invention is a new use of the imaging method using multiple beam interference to create destructive interference in the beam propagation path to reduce the illumination light intensity and so to reduce absorption and scattering of the materials, and to create constructive interference to produce high composite light intensity which forms an inner light layer to illuminate, process the inner object in the materials. The apparatuses are practicable and have great performance. Compared with the traditional apparatuses, for example, the created laser scalpel can treat tissue at depths of more than 5 cm in human body without incision and with high 3D precision of about 1 μm. The effective light energy delivery distance is more than 1000 m in clear seawater.

### FIELD OF THE INVENTION

The present invention relates to laser beam processing apparatus, and more particularly, to the laser beam processing apparatuses and correspondent method using multi-beam interference with or without imaging guidance for light absorption or/and scattering materials..

### BACKGROUND OF THE INVENTION

As a new kind of processing apparatus, the laser beam processing apparatuses are used to denature, vaporize, ablate, etch, weld, drill, and cut various materials with powerful light energy through photo-chemical, photo-ablative, photo-thermal and photo-mechanical effects. The laser beam processing apparatuses have many advantages: including high power density of larger than 100 MW on a area of smaller than 1 mm²; strong ability to treat almost any materials, especially for very hard or very brittle ones; high precision of up to approximate 1 micrometer; fast processing speed of shorter than 1 ns; and less cost for operation and apparatus maintenance to treat some materials compared with using other tools. Therefore, such apparatuses have become important and indispensable equipments in many fields of modern life.

A special advantage of the laser beam processing apparatuses is that the processing can be done without damaging the material surface. For example, the laser beam can selectively etch inner area in a bulk photosensitive glass by focusing the laser beam into the glass. Also, in some laser surgeries, the laser beam can penetrate human skin layer to heat some underneath tissues which absorb light more effectively, such as the tissue containing chromophore, which can induce necrosis of that tissue for treating disease.

This special ability of processing material without damaging material surface, and furthermore, without damaging deeper inner areas in the beam propagation path before the targeted object, is very useful and even indispensable for some applications. It is expected that this special processing ability can be used for more applications and with deeper processing depth.

However, to reach this goal is very difficult. The reason is that most materials, as the optical media, have strong light absorption and scattering, which not only damage inner materials in the light propagation path and attenuate light energy for processing the targeted object, but also produce scattered light to degrade processing precision. In addition, in some applications, the laser beam processing or treating needs imaging the targeted object for guidance, the scattered light will flood the imaging signal light too. These difficulties make laser beam processing can only be used for the inner object in transparent materials like glass, or for inner object with very shallow depth like the tissue under skin layer with depth of less than 3 mm in photodynamic therapy [see reference: T. J. Dougherty, et al, “Photodynamic therapy (Review),” Journal of the National Cancer Institute, Vol. 90, No. 12, 1998, pp.889-905].

The apparatuses, which can reduce light absorption and scattering and deliver the light energy through longer distance in optical media and especially in the turbid optical media with strong light absorption and scattering are extremely valuable and indispensable for some applications. For example, human tissues, water and atmosphere all absorb and scatter light, although their absorption and scattering rates vary large in scale. In the human tissues, as described above, after traveling several millimeters, the light intensity will drop extremely large. In the water, even in the clear seawater, the green light (λ=550 nm), which has good transmittance in the water, can only travel as far as 25 m [W. Hou, “Active Underwater Imaging,” Chapter 4, Ocean Sensing and Monitoring: Optics and Other Methods, SPIE Press Book, 2013, pp.87-93]. It becomes the main difficulty for many underwater applications including underwater light communication and detection. In the atmosphere, if the propagation distance is long, the laser energy will attenuate heavily too, which also produces the difficulty for many applications, including long distance atmosphere light communication, long distance light energy delivery, and so on. In the medical field, especially, if the laser beam can go deeper into the human body without injuring the tissues in the beam propagation path, many diseases can be treated by laser scalpel with high precision, no incision, no bleeding, anti-infection, fast procedure, and moderate cost.

Therefore, the apparatuses which can reduce light absorption and scattering and increase the delivery distance of the light energy in the light absorption and scattering materials are widely and crucially needed in modern societies. It is a challenging task.

### PRINCIPLE OF THE INVENTION

The aim of this invention is to create a new kind of apparatuses which can reduce light absorption and scattering and significantly increase the delivery distance for light energy in the light absorption and scattering materials, that is, in the optical turbid media.

This invention is a new use of an existing invention titled “Method of Inner Light layer Illumination by Multi-beam Interference and Apparatuses for Imaging in Turbid Media”, which is invented by Shangqing Liu, and has been filed for applying U.S. nonprovisional utility patent (application Ser. No.: 17/169,394) on Feb. 6, 2021. In the following, in order to simplify the description, the existing invention titled “Method of Inner Light layer Illumination by Multi-beam Interference and Apparatuses for Imaging in Turbid Media” will be referred to simply as the invention of ILLI.

The inventor of the invention of ILLI only realized, and so described and claimed the usages of that invention for imaging in turbid media (see appendix attached with this specification of the invention). In other words, using the imaging principle of the invention of ILLI to create a new kind of laser beam processing apparatuses is a completely different invention. Under U.S. patent law, the new use for a purpose that is different from what the patent owner contemplated, and if the purpose is sufficiently distinct, the applicant can get a patent for that new use. Therefore, the creative work described here can apply a new patent.

The created laser beam processing apparatuses and correspondent method can simultaneously reduce the absorption and scattering of turbid media greatly, resulting in great increase of light energy delivery distance. The designed apparatuses have excellent performances. By using these apparatuses, the intensity of the delivered light beam can increase more than 14 orders of magnitude than the normal way. The effective delivery distance will reach, for example, more than 500 m in the clear seawater and more than 5 cm in the human body.

In the following, the basic principle of the invented apparatuses and correspondent method will be described. As indicated above, because the existing invention of ILLI will be referred, some words describing this invention will be similar to the words describing the existing invention of ILLI.

The invented apparatuses are designed based on such an idea: in the light beam propagation paths (not only in the beam propagation path to illuminate the object, but also in the signal light return path for imaging the object), the turbid media produce light absorption and scattering, which attenuate the intensities of the illumination and signal beams, and also produce light noises to bury the return signal light. Therefore, one needs to create a apparatus which can make the light beam disappear in the illumination path and so without being absorbed and scattered because light absorption and scattering are directly proportional to the light intensity, and also make the light beam only appear on the object and so only to illuminate and process the object in the turbid media, then the apparatus can further make the reflected signal light disappear in the return path and so without being absorbed and scattered too, and at last only appear at the observation position for observing. When the object is observed, it means that the object is aimed by the light beam, by increasing the power of the light beam to a required value, the object can be processed by illumination light beam. If the intensity of the light beam in the propagation path is still much lower than the intensity of the light beam at the object location, then no light damage or injury in the beam propagation path. Although this unusual laser processing procedure can't be realized in reality, it can be achieved very proximately by using multi-beam interference.

The said method of laser beam processing apparatuses and correspondent method using multi-beam interference comprises: using a negative dispersion generation device to broaden the full width of half maximum of a short light pulse; then making the broadened light pulse enter the material containing the object for processing; also utilizing positive dispersion of the material containing the object to compress the broadened light pulse in the propagation path, and to create a short light pulse again in the material, which forms an inner light layer to illuminate the object; if needed, making the short signal light pulse reflected from the object return along the incident path reversely; during the return path, the full width of half maximum of the short signal light pulse is broadened by positive dispersion of the material again; then, the broadened signal light pulse is compressed by the negative dispersion generation device; and the broadened signal light pulse becomes short signal light pulse again and is received by imaging receiver placed at the observing position for image-guided processing; at last increasing the power of the illumination short light pulse to a required value, the object can be processed.

The method of said invention for laser beam processing using multi-beam interference is described in more detail underneath.

To select N polarized light beams with different angle frequencies ωⱼ (j=0, 1, 2, . . . , N), which have the same or approximately the same amplitudes and the same or approximately the same polarization states. The angular frequency intervals Δω of any two frequency adjacent beams in these N beams are the same or not the same, here supposing the intervals Δω are the same for simplifying the related analyses and calculations. In addition, at a certain moment t the initial phases ϕⱼ (j=0, 1, 2, . . . , N−1) of these N beams are zero. The output beams from mode-locked laser and if their polarization directions are polarized by a polarizer satisfy these conditions [see the reference: P. W. Smith, “Mode-Locking of Laser,” Proc. IEEE, 58(9), 1342-1355, 1970].

The frequency range of these N light beams are in visible region, or/and in infrared, or/and in ultraviolet, or/and in X-ray region(s).

The light fields of these N beams are superimposed to each other on the propagation path first in the negative dispersion generation device and then in the turbid medium containing the object for processing, which produces multiple beam interference. The negative dispersion generation device has negative dispersion and the turbid medium has positive dispersion (in the most situations, the optical media have positive dispersions). Thus, because these beams have different frequencies and different phase velocities in the dispersive medium or device, the destructive interference of the multiple beams makes the composite amplitude of N beams very small in the most of the propagation paths, so the composite light intensity of N beams is attenuated in the propagation path. Generally, the larger the number N, the smaller the composite light intensity of the N beams caused by destructive interference.

The phase differences between any two frequency adjacent beams change gradually in the propagation path. Since the angular frequency intervals Δω between any two frequency adjacent beams are the same, and the initial phases ϕⱼ (j=0, 1, 2, . . . , N) of these N beams are zero at a previous moment, the phase difference of every two frequency adjacent beams changes gradually from zero to the negative value in the negative dispersion generation device first, and then from negative value to zero in the positive dispersion medium next, and at a certain position in the turbid medium, the phase differences of all pairs of two frequency adjacent beams become zero or approximately zero at the same time, resulting in constructive interference of the N beams. That is, the amplitudes of the N beams add to each other coherently, and create composite light intensity maximum. If the number N is large enough and the total spectral width is wide enough, the composite light intensity maximum may become extremely large, and the duration of the composite light intensity maximum may become extremely short. Therefore, a short light pulse, that is, a thin inner light layer in the medium is formed for illuminating and processing the object.

By making the absolute value of the dispersion generated by the negative dispersion generation device be equal to the absolute value of the dispersion generated by the material within the path reaching the object, but with the opposite sign, and making two optical path distances of the output port of the said short light pulse source and the image receiving position to the object in the material be equal, the image receiving position can be determined (see more description below).

Therefore, the said apparatuses and correspondent method will have the following properties:
1. In most sections of the propagation path in the medium, the composite intensity of the N light beams is very small. Thus, the N light beams will not damage the medium material. In addition, because the absorption and scattering of the turbid media are directly proportional to the intensity of the incident light beam, so the absorption and scattering of the turbid media are greatly reduced. This will not only greatly reduce the transmission attenuation of the light energy, leading to more light energy to illuminate and process the object, but also greatly reduce the light noise caused by scattering, thus effectively improving the signal to noise ratio of the object imaging.
2. The object is placed at the position of the composite light intensity maximum. The composite light intensity maximum is usually very large by pulse compression effect. It will be large enough for processing various objects in many different applications. In addition, because the reflected light intensity is proportional to the illuminating light beam intensity, when the illumination light is reflected by the object and becomes the return signal light, the signal light intensity will be further increased largely.
3. The duration of the composite light intensity maximum can be very short, resulting in a very thin inner light layer, which will produce high processing precision. Thus, the laser beam processing has high precision not only on the two-dimensional object plane but also along the longitudinal direction. In addition, thin inner light layer can help to obtain high imaging resolution along the direction of depth of field.
4. The return signal light is still mainly composed of the N incident light beams. Although their amplitudes are significantly or even inconsistently attenuated, and their polarization states are changed or even inconsistently changed, as long as these inconsistencies are not very serious (as in most cases), the return propagation of the signal light from the object may be a completely reverse process of N incident light beams to the object (if the signal light returns along the incident route). Therefore, according to the light ray reversible principle [see the reference: D. S. Goodman, “General principles of Geometric Optics,” Chapter 1, Handbook of Optics, Vol. I, 2ed, Edited by M. Bass, and et al, McGRAW-Hill, New York, 1995, p.1.10], the multiple beam interference can still occur in the return path, resulting in composite light intensity attenuation in most sections of the return path, and composite light intensity maximum appearing at a specific position. This may greatly reduce the absorption and scattering of the turbid media in the return path, preserve the return signal energy and reduce the light scattering noise again. Usually, the imaging receiver is placed at the position of composite light intensity maximum of the return signal light. Sometimes, the signal light travels along a different route from the incident rout to the imaging receiver. Because the required conditions for multiple beam interference may be satisfied too, the destructive interference will also make absorption and scattering of the turbid media small in the signal propagation path, and the constructive interference will make the signal light be received well too.
5. When the number N of the N beams is large enough, or the total spectral width of N beams is wide enough, the composite light intensity maximum can be large enough to process the object in the medium and the composite light intensity in the propagation path can be small enough to not damage the materials.

Generally, the same beam groups having the same characteristics of the said N beams can be repeatedly used to produce a series of composite light intensity maximums which can increase the total energy to illuminate and process the object, and to produce a series of signal pulses which can increase the total energy to be received by the imaging receiver.

The said polarized light beams may be plane polarized, or elliptically polarized, or circularly polarized light beams, because the beams of plane polarized, elliptically polarized and circularly polarized all can produce interference. The said polarization states include polarization directions of the plane polarized light beams, ellipticity of the elliptically polarized light beams. The said N polarized light beams may be plane, or cylindrical or spherical light beams located in the material, the thickness of the layer is much thinner than the processing or imaging distance in the material, or this layer is focused to be point or line located in the material.

Since the plane light beams are used mainly for most applications, the underneath physical analyses and mathematical calculations are based on using plane light beams. For applications of using cylindrical or spherical light beams, the physical analyses and mathematical calculations can follow the similar processes.

When processing the object in the material, the power of the illumination short light pulse is increased to a required value so that the peak intensity of the formed illumination short light pulse is high enough for processing the object. Meanwhile, the light intensity of the illumination light pulse in the propagation path is kept to be low enough for not damaging the material by light pulse broadening.

### BRIEF DESCRIPTION OF THE DRAWINGS

The preferred embodiments of the invented apparatuses will be described underneath. Obviously, these embodiments are not the all apparatuses which can be designed based on the method of this invention. Basing on the method of this invention and using existing technical knowledge, the said apparatus embodiments may be modified and alternated. Therefore, the applicant of this invention reserves the rights of all modifications, alternatives, and equivalent arrangements of the invented apparatus embodiments described underneath.

The aforementioned aspects and advantages of the invention will be appreciated from the following descriptions of preferred embodiments and accompanying drawings wherein:

FIG. 1 illustrates a short light pulse is broadened by positive dispersion medium only (in the top), and is broadened by the negative dispersion generation device first and then is compressed by the positive dispersion medium next (in the bottom).

FIG. 2 illustrates the schematic diagram of optical structure of the medical image-guided no incision laser surgery apparatus.

FIG. 3 illustrates the schematic diagram of optical structure of the underwater image-guided laser energy delivery apparatus.

FIG. 4 illustrates the change of composite light intensity of multi-beam interference in dispersive medium. The light intensity I changes with the parameter K. Not that just taking N=20.

### DETAILED DESCRIPTION OF INVENTED APPARATUSES AND CORRESPONDENT METHOD

The invented apparatuses of laser beam processing for light absorption and scattering materials have been designed based on the method described above. The apparatus comprising: laser generating short light pulse which contains N polarized light beams with different frequencies, the same or approximately the same polarization states, and zero initial phases at a certain moment; the angular frequency intervals Δω of these N beams are equal or not equal but are equal usually; an optical adjusting means to make the amplitudes of the N polarized light beams become the same or approximately the same; a mirrored negative dispersion generation device; an processing or imaging distance adjuster to adjust the processing or imaging distance in the material containing the object; a means to move the formed inner light layer, or light point, or light line in the material in three dimensions.

The short light pulse comes from the mode-locked laser. The same or approximately the same amplitudes of the N polarized light beams is obtained by using dye to make dispersion compensation to laser cavity gain.

Then, the short pulse enters the input surface of a dispersive medium at t=0. Since the pulse contains N frequency components (the number N is from 3 to 10¹² or more), that is, N light beams, the phase difference of any pair of two beams corresponding to the angular frequencies ωⱼ and ωⱼ₋₁ (j=0,1,2,3, . . . , N−1) in these N beams is zero when the pulse enters the input surface of the medium. Also supposing the input surface of the medium is located at the position of x=0, thus, the initial phases φⱼ are zero at x=0 and t=0. The input surface is perpendicular to the x direction.

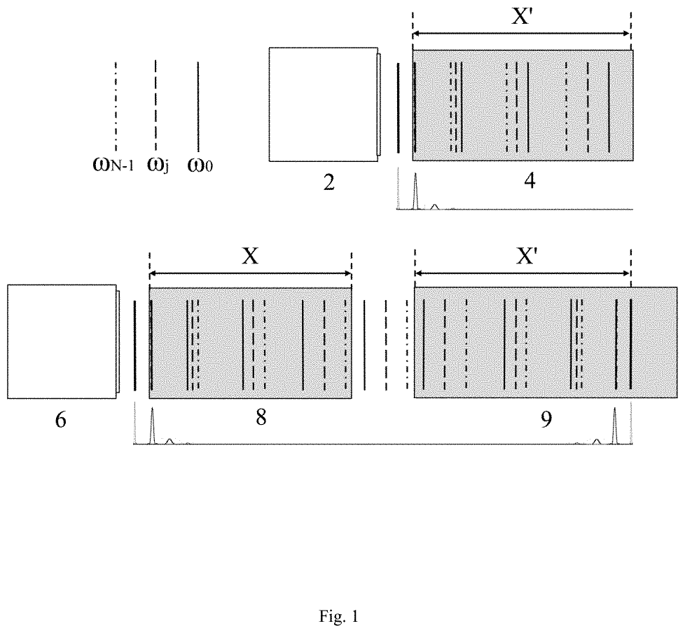

In the most situations, the optical media are positive dispersive media including human body and seawater. When the light pulse enters the positive dispersive medium, the different beams constituting the light pulse travel at different speeds. The higher the beam frequency is, the lower the beam travels. Thus, the pulse broadens and become a beam group with weaker and weaker composite light intensity since destructive interference of the multiple beams as shown in the top of FIG. 1.

In FIG. 1, the light beams are from a mode-locked laser 2. The solid line, dashed line and dotted line represent three planar wavefronts of the beams corresponding to the angular frequencies ω₀, ωⱼ and ω_{N-1}. If these three wavefronts travel in the positive dispersive turbid medium 4, since ω₀<ωⱼ<ω_{N-1}, the wavefront represented by the solid line travels fastest.

When these three wavefronts travel in a negative dispersion generation device 8 (see the bottom of FIG. 1), their traveling speeds are reversed, that is, the higher the beam frequency is, the faster the beam travels. The light beams are from mode-locked laser 6. If the light pulse enters the device 8 at t=0, three wavefronts are overlapped at the position x=0. In the device 8, since ω₀<ωⱼ<ω_{N-1}, the wavefront presented by the dotted line travels fastest.

Thus, the phase difference Δϕⱼ=ϕⱼ−ϕⱼ₋₁ will change with x from zero to negative value although ωⱼ>ωⱼ₋₁. The destructive interference will occur and grow in the device 8 with change of Δϕⱼ from zero to negative value.

Note that the shorter the light pulse duration is, the faster the pulse broadens, and the quicker the pulse peak light intensity decreases. It is because the shorter pulse has wider frequency range and contains more frequency components. If defining the decrease time length of the pulse peak intensity from 100% to a significantly small percentage, such as 0.1%, as the initial broadening period T_{ib}, outside the initial broadening distance D_{ib}=VₐT_{ib}, the light absorption and scattering will become significantly small because the light peak intensity has dropped significantly, where Vₐ is the average speed of the N beams in the dispersive medium. The required initial broadening period T_{ib} or the initial broadening distance D_{ib} depends on the absorption and scattering coefficients of the medium. For the turbid medium with larger absorption or/and scattering, the required T_{ib} or D_{ib} should be shorter. For example, the D_{ib} value should be of the scale of millimeters for medical processing and imaging and of the scale of the meters for underwater processing and imaging. In the same way, the last shortening period Tₗₛ is defined, which is the increase time length of the pulse peak intensity from a very small percentage, such 0.1%, of its maximum value to the 100% of its maximum value. Because the pulse shortening is the reverse process of the pulse broadening completely, T_{ib} should be equal to Tₗₛ in the same optical medium.

After leaving the negative dispersion device 8, the broadened light pulse enters the positive dispersive turbid medium 9. The optical path difference of any pair of two frequency adjacent beams of the pulse decreases gradually in the turbid medium 9. The optical path difference between any two of the three wavefronts shown in FIG. 1 also decreases gradually. Thus, in other words, the light pulse broadening terminates, and the light pulse shortening begins.

Let the phase difference between two beams corresponding to angular frequencies ωⱼ and ωⱼ₋₁ in the turbid medium be Δϕ′ⱼ, the propagation distance of the beam corresponding to angular frequency ωⱼ be x′ⱼ in the turbid medium, and the refractive indexes of the turbid medium corresponding to the angular frequencies ωⱼ and ωⱼ₋₁ be n′ⱼ and n′ⱼ₋₁. Then, if the medium used in the device for generating the negative dispersion is the same medium as the turbid medium, or it has the same or very approximate dispersion property as the turbid medium, then, n′ⱼ=nⱼ and n′ⱼ₋₁=nⱼ₋₁. Under this condition, if taking x′ⱼ=xⱼ, because Δϕⱼ is produced by negative dispersion generation device, we get Δϕ′ⱼ=−Δϕⱼ. Thus, because Δϕ′ⱼ−Δϕⱼ=0 for every pair of two frequency adjacent beams, the broadened light pulse will be compressed completely. A thin inner light layer will be formed in the turbid medium 9. Therefore, by making mirrored negative dispersion compensation, the expected thin inner light layer can be formed in the turbid medium (please see more detailed descriptions in the attached appendix).

The said light absorption and scattering materials include human body, animal body, seawater, river water, lake water, pond water, fog, smog, snow, ice, cloud, atmosphere and any gaseous, liquid or solid materials which have light absorption or/and scattering, especially have strong light absorption or/and scattering.

The said laser beam processing includes medical laser beam treatments, medical laser beam surgery, light communications in atmosphere or water, various light energy delivery in bulk gas, bulk liquid and bulk solid materials for heating, denaturing, ablating, etching, welding, drilling, vaporizing, hitting, cutting, destroying, and so on

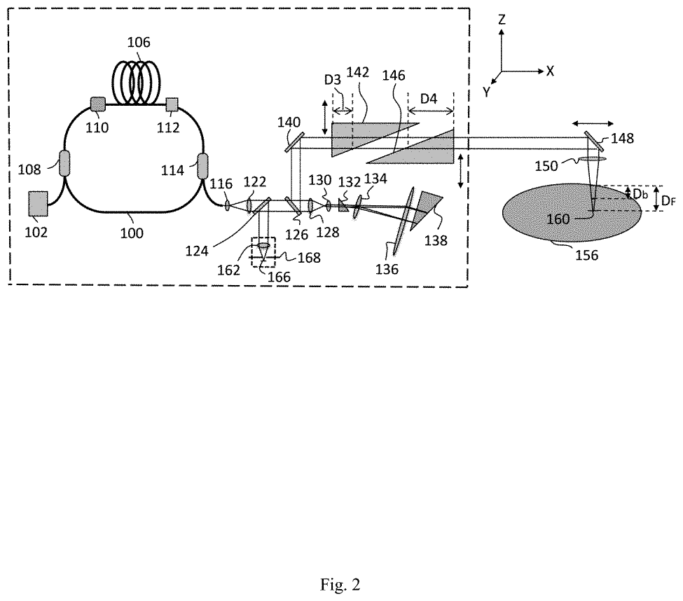

The medical image-guided no incision laser surgery apparatus is described below as the first preferred embodiment of the apparatuses according to the invention. FIG. 2 is the schematic diagram of this apparatus optical structure. Because the sizes of various parts and components differ largely, in order to show necessary details, the shown structure is not drawn in actual proportion.

The short light pulse comes from a mode-locked fiber laser 100, which is pumped by a light-emitting diode 102. The pump light enters a doped fiber 106 through a coupling element 108. When the total spectral bandwidth of the laser output beams needs to be wide, several light-emitting diodes with different emitting frequencies may be used jointly to pump the fiber 106. An optical isolator 110 and a polarization controller 112 are used to ensure unidirectional beam oscillation. The mode-locked fiber laser with multi-wavelength output has been developed maturely [see the references: N. Li, et al, “Cavity-length optimization for high energy pulse generation in a long cavity passively mode-locked all-fiber ring laser,” Applied Optics, 51, 17, 2012, pp.3726- 3730].

The laser beams go out through an optical coupler 114, and then the beam diameters are enlarged by lens 116 and 122. After passing through the beam splitter 124, 10% of the light energy enters the second beam splitter 126. The transmittance of the beam splitter 126 is 50%. Then, 5% of the total incident light energy enters two lenses 128 and 130 for beam shrinking and focusing. To use a beam splitter 124 with low transmittance is for less signal light energy loss when the signal light is reflected by beam splitter 124 later. Then, the shrank and slight focused beams enter a right-angle prism 132 normally.

The vertex angle of the prism 132 is β, therefore, the incident angles of the central lines of the N thin and slight focused beams to the output surface of the prism 132 are β too. When these beams are refracted by the prism 132, the refractive angle θⱼ of the central line of the beam corresponding to angular frequencies ωⱼ is [see the reference: D. S. Goodman, “General principles of Geometric Optics,” in Handbook of Optics, McGRAW-Hill, 1995, Vol. I].

nⱼ sin β=sin θⱼ, (1)

where nⱼ is the refractive index of the prism 132 corresponding to the angular frequency ωⱼ, and the refractive index of atmosphere is approximately 1. After being refracted by the prism 132, all beams enter a thin lens 134. f₃ is the focal distance of lens 134. In order to simplify the analyses and calculations, the central line of the beam corresponding to the angular frequency ω_{N-1} is arranged along the optical axis of the lens 134 and through the center of the lens 134. U is the distance from the beam centers on the output surface of the prism 132 to the center of the lens 134. If U<f₃, according to Newton equation for thin lens [see the reference: D. S. Goodman, “General principles of Geometric Optics,” in Handbook of Optics, McGRAW-Hill, 1995, Vol. I], the angle of θ_{N-1}−θⱼ, which is the angle between two central lines of the beams corresponding to the angular frequencies ω_{N-1} and ωⱼ, is magnified by M times to θ′ⱼ, that is

θ′ⱼ=M(θ_{N-1}=θⱼ). (2)

Where M=f₃/(U−f₃). When M is negative, the image is a virtual image.

After the refractive angles are amplified, the N beams enter thin lens 136 all along the directions parallel to the optical axis of the lens 136. f₄ is the focal distance of the lens 136. Then, the N parallel beams all enter prism 138. The input surface of the prism 138 is planar and perpendicular to the optical axis of the lens 136.

In the prism 138, the height of the central line of the beam corresponding to the angular frequency ωⱼ is Hⱼ (from the optical axis of the lens 136), the travel distance of the beam corresponding to the angular frequency ωⱼ is Dⱼ. From FIG. 2 (see more descriptions in the attached appendix),

$$\frac{{H}_{j}}{{f}_{4}} = tg {θ}_{j}^{′} . \qquad ( 3 )$$

When the beams travel a distance D in the prism 138, two beams corresponding to the angular frequencies ωⱼ and ωⱼ₋₁ will produce an optical path difference ΔPⱼ as

ΔPⱼ=DΔnⱼ, j=0,1,2,3, . . . N−1, (4)

where

Δnⱼ=nⱼ₋₁−nⱼ. (5)

If the positive dispersion generated in the turbid medium needs to be compensated ideally, all optical path differences produced by all pairs of two frequency adjacent beams in the turbid medium must be generated in the prism 138 equally but with the opposite signs, that is, a mirrored negative dispersion must be generated.

In order to generate mirrored negative dispersion, the prism 138 must be made of the same medium as the turbid medium, or the prism 138 must have the same or very approximate dispersion property as the turbid medium. To satisfy such a requirement has become relatively easy in recent years. For example, to find a material whose optical property is approximate to the human tissues is not difficult because of the development of tissue simulating phantoms [see the reference: B. W. Pogue, and M. S. Patterson, “Review of tissue simulating phantoms for optical spectroscopy, imaging and dosimetry,” J. Biomed. Opt., 11(4), 02.1-02.16, 2006]. In the optical spectroscopy, imaging, and therapy research fields, such simulating materials have been widely used. The dispersion, absorption and scattering properties of these materials are characteristic of human tissues. Of course, to choose the material to make the prism 138, its light absorption and scattering should be small for saving light energy. If the chosen material is soft, the prism 138 may be made to be a transparent container and filled with the chosen material.

Suppose the optical path difference produced by two beams corresponding to the angular frequencies ωⱼ and ωⱼ₋₁ in the turbid medium is ΔP′ⱼ=D′Δn′ⱼ, where Δn′ⱼ=n′ⱼ−n′ⱼ₋₁, and n′ⱼ and n′ⱼ₋₁ are the refractive indexes of the turbid medium corresponding to the angular frequencies ωⱼ and ωⱼ₋₁. D′ is the traveling distance of the beam corresponding to the angular frequency ωⱼ in the turbid medium (D′ may be regarded as the processing or imagine distance in the turbid medium). After satisfying the requirement for medium dispersive property, the negative dispersion generation device needs to generate the following optical path differences

ΔPⱼ=−ΔP′ⱼ=−D′Δnⱼ, j=0,1,2,3, . . . , N−1, (6)

In addition, except for the prism 138, other optical elements used in the negative dispersion generation device also produce positive dispersions. If the total value of the traveling path lengths of the beams in these elements is much less than D′, these additional positive dispersions can be ignored. Otherwise, they need to be compensated too. Usually, all of the optical elements used in the negative dispersion generation device should be made of the same medium or have the same or very approximate dispersive property as the turbid medium.

Because the prism 138 can only be made from positive dispersive medium, to produce negative optical path difference for any pair of two beams corresponding to the angular frequencies ωⱼ and ωⱼ₋₁ when ωⱼ>ωⱼ₋₁, the only way is to change the propagation distance difference of any pair of two beams in the prism 138.

If the propagation distances of the two beams corresponding to the angular frequencies ωⱼ and ωⱼ₋₁ are Dⱼ and Dⱼ₋₁, respectively, Dⱼ and Dⱼ₋₁ must satisfy the condition

Dⱼnⱼ−Dⱼ₋₁nⱼ₋₁≈ΔDⱼnⱼ=ΔPⱼ=−ΔP′ⱼ=−D′Δnⱼ, j=0,1,2,3, . . . , N−1. (7)

Where ΔDⱼ=Dⱼ−Dⱼ₋₁.

Rearranging Eq.(7), we have

$$Δ {D}_{j} = - {D}^{′} \frac{Δ {n}_{j}}{{n}_{j}} = {D}^{′} \frac{{n}_{j - 1} - {n}_{j}}{{n}_{j}} , \\ j = 0 , 1 , 2 , 3 , … , N - 1. \qquad ( 8 )$$

Eq. (8) gives the negative optical path difference required for two beams corresponding to the angular frequencies ωⱼ and ωⱼ₋₁ in the prism 138. Thus, the total required propagation distance Dⱼ of the beam corresponding to the angular frequency ωⱼ in the prism 138 is

Dⱼ=ΔD_{N-1}+ΔD_{N-2}+ΔD_{N-3}+ . . . +ΔDⱼ. (9)

In order to produce mirrored negative dispersion compensations, a method has been crated. It is based on using computer-controlled high precision optical machining and retroreflective micro-mirrors.

The method is accomplished by measuring the turbid medium refractive indexes corresponding to different frequencies within the required range first. Because the number of the frequencies is large, only partial and discrete data need to be measured. Then, one can use a computer to fit refractive index change curve with frequency from the obtained data. There are several dispersion equations for fitting the refractive index change curves, such as Cauchy, Hartmann, Conrady and Kettler-Drude equations, etc. [see the reference: W. J. Smith, “Optical Materials and Interference Coatings,” in Modern Optical Engineering, McGRAW-Hill, 2000, Chapter 7, p. 176].

Then ΔDⱼ can be calculated from the fitted refractive index curve and the required D′ value according to Eq. (8). The D′ is the expected processing or imaging distance in the turbid medium. At last, the computer is used to obtain the total propagation distance Dⱼ for each beam corresponding to the angular frequency ωⱼ by Eq.(9).

From Eq.(2) and Eq.(3), we have

Hⱼ=f₄tg[M(θ_{N-1}=θⱼ)]. (10)

From Eq.(1), we have θⱼ=nⱼ arcsin β and θ_{N-1}=n_{N-1} arcsin β. Thus,

Hⱼ=f₄tg[M(n_{N-1} arcsin β−nⱼ arcsin β)]. (11)

Using Hⱼ value as the position for a point along the direction perpendicular to the optical axis of the lens 136, and using Dⱼ value corresponding to that Hⱼ as the position for that point along the direction of the optical axis of the lens 136, thus, a data group that contains N data pairs for N points can be produced in the same way. Then, the computer is used to fit a smooth curve which connects the N point positions of (Dⱼ, Hⱼ) from the produced data group. At last, the computer-controlled high precision grinding and polishing are used to shape the output surface of the prism 138 according to the fitted smooth curve.

In recent years, the computer-controlled high-precision optical grinding and polishing have been developed significantly, which can fabricate the optical elements with very high accuracy [see the reference: D. W. Kim, H. M. Martin, and J. H. Burgea, “Calibration and optimization of computer-controlled optical surfacing for large optics,” Proc. SPIE, 8126, 15.1-15.10, 2011].

When the N beams are incident on the output surface of the prism 138, because the output surface shape is generally non-planar and non-spherical, the incident angles of the N beams are different. Especially, since the incident beams are focused beams, even the light rays of each beam have different incident angles. Therefore, these beams can't return back along their incident paths by simply making the prism output surface become a reflective surface.

This problem can be solved by using retroreflective micro-prism mirrors. In recent years, the micro-prisms are used as the tiny tetrahedrons and are placed in arrays on thin hard or soft sheet surface. These retroreflective sheets can reflect light beams within wide spectral range. When the beam incident angle is less than 30°, the reflectivity can be >90%. The average diameter of these micro-prisms is less than 45 μm [see the reference: A. Lundvall, F. Nikolajeff, and T. Lindstrom, “High performing micromachined retroreflector,” Opt. Express, 11(20), 2459-2473, 2003; A. Poscik, J. Szkudlarek, and G. Owczarek, “Photometric properties of retroreflective materials in dependence on their structure and angle of illumination,” Fibres Text. East. Eur. 3(135), 58-64, 2019].

When a soft micro-prism retroreflective mirror layer is pasted on the smooth output surface of the prism 138 by optical glue, the focused N beams with different incident angles can be returned to travel along their incident paths completely. Note that the returned beams will travel along their previous path once more, so D′ in Eq.(8) should be reduced to 0.5 D′.

The returned N beams from the prism 138 recombine in the prism 132. After being reflected by beam splitter 126 and mirror 140, these beams become parallel beams and enter an imaging distance adjuster consisting of two triangular components 142 and 146. Because the length and shape of the prism 138 have determined a fixed processing or imaging distance D′ in the turbid medium by the Eq.(8) and Eq.(9), every apparatus has a fixed imaging distance D′. Therefore, if the expected processing or imaging depth in the human body is D_{b}, the distance changes by adjusting two components 142 and 146 are D₃ and D₄, then to make

D′=D₃+D₄+D_{b}. (12)

The expected processing or imaging depth D_{b} can be adjusted by changing D₃+D₄. Note that the two components 142 and 146 should be made from the same medium as the turbid medium or have the same or very approximate dispersive property as the turbid medium too. Because two triangular components have symmetrical shapes, no unwanted dispersions will be produced by the distance adjuster.

Then, the N parallel beams enter the human body 156 by reflection of the mirror 148. If there is no lens 150, an inner light layer will be created at the depth of D_{b}. The layer thickness is determined by two factors. One is the number N of the beams and the frequency interval Δν(2πΔν=Δω) of the N beams according to the relation of NΔνΔτ=1 [see the reference: W. H. Carters, “Coherence theory,” in Handbook of Optics, McGRAW-Hill, 1995, Vol. I, p.4.3], where Δτ is the duration of the light pulse when Δϕⱼ=0, which determines the inner light layer thickness δH by δH=VₕΔτ, where Vₕ is the average speed of the N beams in the human body. If the total spectral width of the N beams is wide enough, the layer thickness can be very thin, such as less than 1 μm. Another factor is the initial broadening period T_{ib}. The pulse broadening due to the chromatic dispersion can be estimated as [see the reference: C.-A. Bunge, M. Beckers, and B. Lustermann, Polymer Optical Fibres, Fibre Types, Materials, Fabrication, Characterization and Applications, Elsevier Ltd, Woodhead Publishing, 2017, pp.47-118]

ΔT′=L′Δλd_{c}, (13)

where Δλ is the pulse spectral width in wavelength, d_{c} is chromatic dispersion coefficient, and L′ is the propagation distance of the pulse in the dispersive medium, ΔT′ is full width of half maximum (FWHM) of the pulse. For example, the typical value of d_{c} is 20 ps/nm·km at 1550 nm for telecom fibers. Thus, if Δλ=1000 nm, which corresponds to 2 fs light pulse, when L′=1 mm, ΔT′=20 fs. Because 2 fs pulse broadens to 20 fs, the peak light intensity of the pulse should drop to below 10% of its maximum value. For seawater, typical d_{c} values are from 60 ps/nm·km to 300 ps/nm·km [see the reference: “Seawater intrusion and mixing in estuaries,” Marine Species Introduced Traits Wiki, 2020, marinespecies.org/introduced/wiki/Seawater_intrusion_and_mixing_in_estuaries#Experimental_determination_of_the_longitudinal_dispersion_coefficient]. Considering that about 60% of human body is water by weight, thus roughly speaking, the ultra-short light pulse of fs level can broaden fast enough in the human body too (the dispersion coefficients of the human tissues have not been found temporarily). Therefore, if a pulse of fs level broadens by negative dispersion first, then it will shorten fast enough in the human body during the last shortening period Tₗₛ. Thus, the light energy loss due to light absorption and scattering during the last shortening period Tₗₛ is small. Fortunately, obtaining ultrafast, high power fs lasers is not difficult nowadays.

In FIG. 2, the N beams are focused by lens 150 when they enter the human body 156. It is for confocal imaging to improve the imaging longitudinal resolution, which will be explained later.

The signal light beams reflected by the targeted tissue return along the incident path reversely. Generally the N beams constituting the incident pulse will all be reflected by the target tissue. The reflections occur on the interface on the targeted tissue surface and between the two areas with different refractive indexes. The reflectivities of the interface for N beams don't differ much usually. During the return path, the signal light pulse will broaden by positive dispersive tissues again as the signal light pulse still contains multiple frequency components, that is, the multiple beams, which results in decrease of the composite intensity of signal light beams, and so results in decrease of the light absorption and scattering in the body again. Then, the signal light beams exit the body. The optical path differences of the signal light beams are further enlarged by positive dispersive imaging distance adjuster. After reflected by beam splitter 126, the signal light beams enter the prism 138 again. This time, the broadened signal pulse will be compressed by negative dispersion by the prism 138. Because the return process of the signal pulse is a completely reverse process of the laser pulse illumination process, the detailed mathematic description does not needed.

When the signal beams reach the beam splitter 124 again, the signal light beams travel a distance which equals D′ exactly. Thus, the expected signal pulse appears by constructive interference of N signal beams. After reflected by beam splitter 124, as beam splitter 124 has high reflectivity, most of the energy of the signal light pulse is focused on the image plane 166 by lens 162.

If the lens 150 is not used, the designed apparatus has the most popular imaging structure, which can make one point on the object plane become one point on the image plane directly. This structure can easily combine existing ultra-resolution technologies [see the reference: G. Huszka, and M. A. M. Gijs, “Super-resolution optical imaging: A comparison,” Micro and Nano Eng. 2, 7-28, 2019], such as to place a phase filter before the focusing lens 162. In this way, the imaging resolution along the object plane can exceed the theoretical diffraction limit, which is significantly less than the beam wavelengths.

Using the lens 150 is for improving the longitudinal resolution. Because during the last shortening period Tₗₛ in the human body, the light pulse intensity will be significantly large. For example, as described above, within the range of 1 mm, the intensity of a 2 fs pulse is about 10% of its maximum value in the telecom fiber. As the initial broadening period and the last shortening period has equal length in the same medium, the effective thickness of a 2 fs pulse will be much larger than its theoretical thickness of approximate 0.2 μm in the human body, which will reduce the longitudinal resolution of the imaging. The confocal imaging can solve this problem [see the reference: S. Inoue, and R. Oldenbourg, “Microscopes,” in Handbook of Optics, McGRAW-Hill, 1995, Vol. II]. By using lens 150 to focus the illumination light beams to scan the targeted tissue, and using a spatial pinhole 168 placed before the image plane 166 to block out-of-focus light in image formation, the imaging longitudinal resolution can be increased to wavelength level, that is, about 1 μm, and with better contrast.

When using the lens 150, the focal point of the lens 150 is at the position with the depth of D_{F} in the human body. It should be indicated here, the formed inner light layer and the focal point 160 locate at different positions, which gives a special benefit of more conveniently changing the processing area size and controlling the processing power density, because the area size, and so the power density of the formed inner light layer can be also changed by adjusting the distance difference of D_{F}−D_{b}.

This apparatus creates a thin inner light layer in the human body 156 in the approximate 2D XY plane. The layer area size may also be changed by the distance difference of D_{F}−D_{b}. In contrast to the focal point scanning illumination, which is used by many existing 3D imaging or medical treating technologies, this 2D illumination simplifies the apparatus optical structure and improves the imaging and treating speed.

The mirror 148 can move in the X direction. The apparatus or the human body 156 can move in the Y direction. Thus, by adjusting the processing or/and imaging depth D_{b}, the 3D imaging or treating can be achieved in the human body 156. The change of the distance difference of D_{F}−D_{b} is by moving the lens 150 in the Z direction.

Many medical treatments need imaging the targeted tissue for guidance. This apparatus has excellent imaging ability. The signal light reflected by the target tissue returns along the incident path reversely. The returned signal light is almost entirely consisted of the previous illumination N beams with reduced amplitudes and somewhat changed polarizations. In the returning path, because the returned beams have different frequencies and different phase velocities, thus, the destructive interference makes the composite light intensity small.

When the targeted tissue is observed clearly by observer, it also means that the targeted tissue is aimed by the laser beam exactly. Then by raising the output power of the mode-locked laser to the required level for treating the targeted tissue, the targeted tissue can be vaporized, or ablated, or incised by formed inner light layer, which may be called as laser scalpel. When the illumination light power is raised for treating the tissue, the light intensity in the beam propagation path can be still under the safe threshold value since multiple beam interference (see further descriptions below). Afterwards, by reducing the output power of the mode-locked laser to previous level, the result of such no incision laser surgery can be checked by imaging the targeted tissue again.

The method of said invention may further combine a variety of existing technologies to produce a variety of laser processing with or without image guidance. As these are existing technologies and knowledge, no further explanation is needed too.

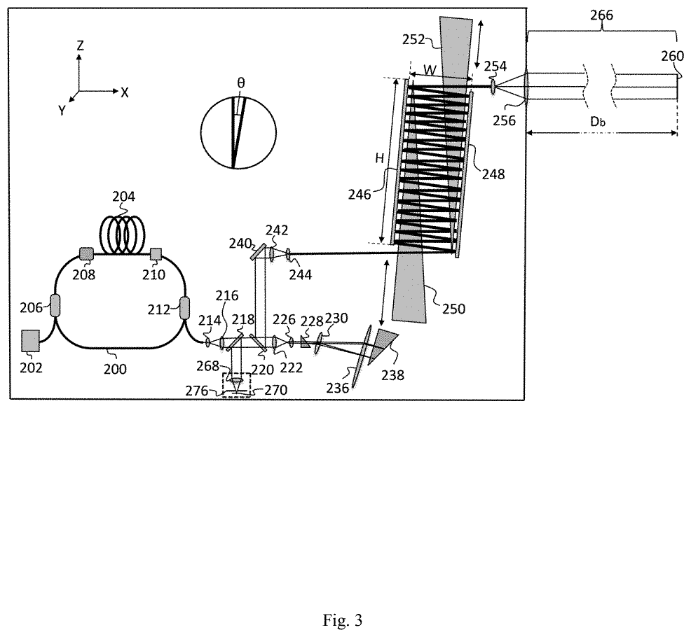

The underwater image-guided laser energy delivery apparatus is described below as the second preferred embodiment of the apparatuses according to the invention. FIG. 3 is the schematic diagram of this apparatus optical structure. Because the sizes of various parts and components differ largely, in order to show necessary details, the shown structure is not drawn in actual proportion too.

In FIG. 3, the mode-locked fiber laser 200 consists of light-emitting diode 202, doped fiber 204, coupling element 206, optical isolator 208, polarization controller 210 and optical coupler 212. The laser beam diameters are enlarged by lens 214 and 216. After passing through beam splitters 218 and 220, the laser beams enter negative dispersion generation device consisting of lenses 222, 226, 230 and 236, prisms 228 and 238. In the prism 238, the negative dispersion is produced as that in the above described medical laser surgery apparatus.

The processing or/and imaging distance adjuster is composed of two paralleled mirrors 246 and 248, and two triangular components 250 and 252. Two mirror planes are inclined at an angle θ_{M} to Z axis.

Since the desired processing or/and imaging distance underwater is long, after N parallel beams entering the distance adjuster, each beam will be reflected multiple times in the adjuster. Making the diameter of each beam be small, thus each beam can obtain a larger number of reflections between two mirrors. If the expected processing or/and imaging distance in the seawater 266 is D_{b} , the distance change by adjusting the components 250 and 252 is ΠD_{w}, and the designed processing or/and imaging distance of the apparatus is D′. By making D′=D_{b}+ΠD_{w}, the D_{b} can be adjusted by changing ΠD_{w}. Here, Π is the number of the reflection times of a beam between two mirrors, D_{w} is the travelling distance of a beam in two components between two reflections. Because the diameters of the beams are small, such as a diameter of 5 mm, the required thickness of the imaging distance adjuster is thin, such as less than 10 mm, so the distance adjuster can have moderate volume and light weight. Furthermore, if required, the distance adjuster can be a composite distance adjuster composed of multiple distance adjusters.

The change of the value of ΠD_{w} is by moving the components 250 and 252 simultaneously along the mirror planes in the opposite directions. Moving the components 250 and 252 simultaneously and in the opposite directions is for offset extra dispersions caused by the triangular shapes of two components.

After the diameters of the N beams are expanded by lens 254 and 256, the N parallel beams enter the seawater 266 and form an inner light layer on the object plane 260 at the position with the distance of D_{b}.

This apparatus forms a 2D illumination, which will simplify the imaging optical structure and improve imaging speed, since a 2D inner light layer is formed in the YZ plane in the seawater 266. The layer area is determined by the cross-sectional area of the N beams group. The Group of the N beams can scan up and down, from the right to the left, in order to achieve large range 3D illumination and energy delivery in the seawater 266.

Compared with the medical imaging and treating apparatus, the depth resolution requirement for the underwater imaging and delivery is generally much lower than that for the medical imaging and treating. In the seawater, the expected imaging and delivery distance is from several meters to even kilometers, thus the depth resolution of 0.1 mm to 1 mm is very enough generally, which is 2 to 3 orders of magnitude lower than the requirement for medical apparatus. Therefore, the total spectral bandwidth of the N beams is 2 to 3 orders of magnitude narrower than that of the medical apparatus too.

The signal light produced by reflection from the object in the formed light layer returns back along the incident path reversely, and going through a process similar to that of the medical imaging described above. At last, the signal light beams are reflected by the beam splitter 218 and create the expected signal light pulse, which is focused on the image plane 270 by lens 268.

In the same way, in order to improve the longitudinal resolution of imaging, a spatial pinhole 276 is placed before the image plane 270 to form the confocal imaging.

The lens 268 and the 2D image plane 270 also form the most common camera structure, which makes one object point become one image point, and so it is easy to get high imaging resolution and fast imaging speed.

The returned signal light rays from the points of the object plane are not drawn in FIG. 3. In addition, the parallel illumination light beams may be focused on the object plane, then by point scanning way to produce the image on the image plane 270 or to focus the light energy to a tiny point on the object plane.

Also when the object in the seawater is observed clearly by observer, it means that the light energy can be delivered to that object by raising or not raising the output power of the mode-locked laser. For example, the underwater light communication may not need to raise the laser beam power. Some other applications, such as to hit shark or other dangerous creatures needs to raise the laser beam power. If the laser beam power is raised to the level high enough, more objects including non-life ones can be processed or destroyed too. Afterwards, by reducing the output power of the mode-locked laser to the imaging level, the result of light energy delivery can be checked by imaging the object.

Similarly, based on the method of the invention, a variety of existing technologies can combined to create a variety of new functions for underwater apparatus. Since these works may be done by using the existing knowledge, no more descriptions are given here.

### Brief Description of Performances of the Invented Apparatuses

In the following description, the imaging and laser beam processing performances and signal enhancement by using the said apparatuses are given.

Following the way of the analyses and derivations described in the existing invention titled” Method of inner light layer illumination by multi-beam interference and apparatuses for imaging in turbid media” (see the attached appendix), we have the composite light intensity of the multi-beam interference of the N beams in dispersive medium is

$$I = {A}^{2} ( x ) \frac{{cos}^{2} [ \frac{( N - 1 ) ( K Δ ω )}{2} ] {sin}^{2} ( \frac{NK Δ ω}{2} )}{{sin}^{2} ( \frac{K Δ ω}{2} )} . \qquad ( 14 )$$

where K=t−(2xn₀/C), A is the amplitude of the N beams (supposing the amplitudes of the N beams have the same value), t is the time, x is the beam traveling distance in the turbid medium, n₀ is the refractive index of the turbid medium corresponding to the angular frequency ω₀, C is light speed in vacuum.

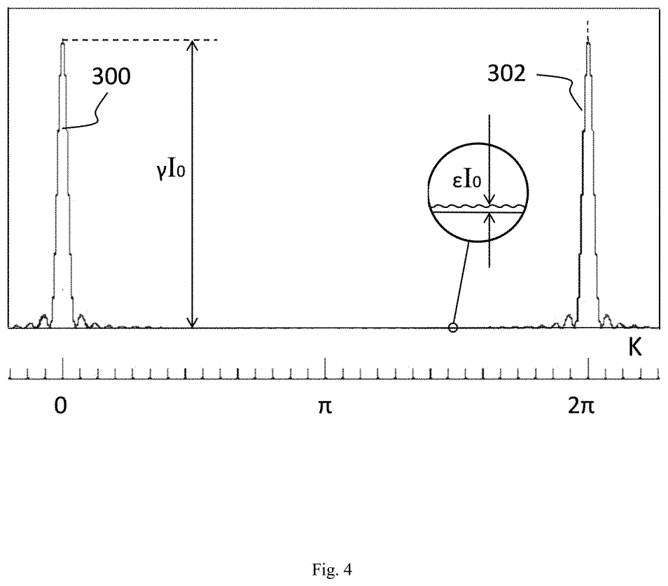

When KΔω becomes zero, the value of I goes to the maximum. The results of numerical calculations by Eq.(14) are shown in FIG. 4 and Table 1. In FIG. 4, I changes with parameter K. 300 and 302 are two composite light intensity maximums. Using K as the unit of transverse coordinate is for avoiding complicated theoretical derivations, and the essential characteristics of the multi-beam interference in optical dispersive medium can still be shown. The I curve in FIG. 4 may be regarded as the change of the composite light intensity of N beams with x at the moment when the inner light layer is recreated. In the calculations, A=1. In order to show the width of the composite light intensity maximum obviously, just taking N=20.

In the Table 1, N is the number of the beams participating in the interference. γ is the enhancement factor of the composite light intensity maximum γI₀. ε is the attenuation factor of the remaining composite light intensity εI₀ between two composite light intensity maximums (see FIG. 4). I₀ is the incident intensity of each beam of the N beams. We see that γ increases with N increase rapidly (see attached appendix for the detailed descriptions about the calculations).

TABLE 1
Signal Intensity Enhancement at Different Imaging Depth in Human Body by Multi-beam Interference.

| N | γ | ε | Factor | D′ = 2 cm | D′ = 5 cm | D′ = 10 cm | D′ = 15 cm | D′ = 20 cm |
|---|---|---|---|---|---|---|---|---|
| 1.00E+01 | 1.00E+02 | 5.05E−14 | ξ | 1.10E−01 | 1.10E−01 | 1.10E−01 | 1.10E−01 | 1.10E−01 |
| 1.00E+02 | 1.00E+04 | 6.11E−10 | ξ | 1.10E+01 | 1.10E+01 | 1.10E+01 | 1.10E+01 | 1.10E+01 |
| 1.00E+03 | 9.97E+05 | 6.21E−06 | ξ | 1.10E+03 | 1.10E+03 | 1.09E+03 | 1.09E+03 | 1.09E+03 |
| 1.00E+04 | 7.08E+07 | 2.80E−06 | ξ | 7.79E+04 | 7.79E+04 | 7.78E+04 | 7.78E+04 | 7.78E+04 |
| 1.00E+05 | 7.08E+09 | 1.13E−05 | ξ | 7.78E+06 | 7.78E+06 | 7.76E+06 | 7.75E+06 | 7.74E+06 |
| 1.00E+06 | 7.08E+11 | 9.35E−06 | ξ | 7.79E+08 | 7.78E+08 | 7.77E+08 | 7.76E+08 | 7.75E+08 |
| 1.00E+07 | 7.08E+13 | 5.31E−06 | ξ | 7.79E+10 | 7.78E+10 | 7.78E+10 | 7.77E+10 | 7.77E+10 |
| 1.00E+01 | 1.00E+02 | 5.05E−14 | α (dB) | 2.63E+02 | 6.27E+02 | 1.23E+03 | 1.84E+03 | 2.45E+03 |
| 1.00E+02 | 1.00E+04 | 6.11E−10 | α (dB) | 2.83E+02 | 6.47E+02 | 1.25E+03 | 1.86E+03 | 2.47E+03 |
| 1.00E+03 | 9.97E+05 | 6.21E−06 | α (dB) | 3.03E+02 | 6.67E+02 | 1.27E+03 | 1.88E+03 | 2.49E+03 |
| 1.00E+04 | 7.08E+07 | 2.80E−06 | α (dB) | 3.21E+02 | 6.85E+02 | 1.29E+03 | 1.90E+03 | 2.51E+03 |
| 1.00E+05 | 7.08E+09 | 1.13E−05 | α (dB) | 3.41E+02 | 7.05E+02 | 1.31E+03 | 1.92E+03 | 2.53E+03 |
| 1.00E+06 | 7.08E+11 | 9.35E−06 | α (dB) | 3.61E+02 | 7.25E+02 | 1.33E+03 | 1.94E+03 | 2.55E+03 |
| 1.00E+07 | 7.08E+13 | 5.31E−06 | α (dB) | 3.81E+02 | 7.45E+02 | 1.35E+03 | 1.96E+03 | 2.57E+03 |

The calculation results shown in FIG. 4 and Table 1 indicate that the larger the N value, the higher the light peak intensity γI₀, the narrower the full-width of half maximum of the light peak intensity γI₀. At the same time, the larger the N value, the smaller the remaining light intensity εI₀ between two peak light intensities.

The numerical calculations show that when the N changes from 10¹ to 10⁷, the enhancement factor γ of the peak intensity γI₀ changes from 10² to 10¹⁴, and the attenuation factor ε of the remain light intensity εI₀ changes from 10⁻¹⁴ to 10⁻⁶. The calculation results are shown in Table 1.

In the Table 1, the difference between the composite peak light intensity γI₀ and the remaining composite light intensity εI₀ may be over 18 orders of magnitude. Such large intensity difference can certainly give plentiful room to avoid light injury for human tissues or light damage for materials in the laser beam propagation path. In the medical surgery applications, the light power density of less than 10 mW/cm2 is safe for human tissues including skin [see reference: T. J. Dougherty, et al, “Photodynamic Therapy (Review),” Journal of the National Cancer Institute, Vol. 90, No. 12, 1998, pp.889-905]. And the power density of higher than 10 W/cm2 can ablate most targeted tissues in human body without problem. The difference between 10 mW/cm2 and 10 w/cm2 is just 3 orders of magnitude. Therefore, no incision laser surgery can be completed by the invented apparatus. For the underwater light energy delivery, hitting a shark perhaps needs a light power density of about 100 W/mm2 because that a light power density of 500 W/mm2 can cut a steel plate [see reference: Miyamoto and H. Maruo, “Mechanism of laser cutting,” Welding in the World, Le Soudage Dans Le Monde, Vol. 29, No. 9/10, 1991, pp.283-294]. Therefore, if the light power density of the laser beam is 50 mW/mm2 in the propagation path, which should not heat the water obviously. The difference between 50 mW/cm2 and 500 W/cm2 is 4 orders of magnitude. Therefore, good underwater light energy delivery can also be completed by the invented apparatus.

Now, based on the actual absorption and scattering situations of the seawater and human body, the signal intensity change and imaging sensitivity enhancement of the said apparatuses can be calculated. In the human body, the absorption coefficient and scattering coefficient are different for different tissues. Here, the average absorption coefficient μ_{ba}=0.397 mm⁻¹ and scattering coefficient μ_{bs}=1 mm⁻¹ of the human blood are taken for the whole body temporarily [see above Dr. M. C. Hillman's doctoral thesis]. Although taking the coefficients of blood for whole human body is differ from the actual situation, as mentioned above, since there is a large amount of blood in the human body and the coefficients of the blood have approximate order of magnitude as those of the most human tissues, such treatments can give approximate results. In addition, there is a practical method for determining the expected processing or imaging distance in the media consisting of the compositions with different refractive indices. it will be given at the last of this invention. The light reflectance R is assumed for the case that light is reflected from the interface between the blood and adipose. The refractive indices of the blood and adipose are taken as 1.3771 and 1.4714, respectively (correspondent to the wavelength of 680 nm). Then, according to the Fresnel formula, at the boundary of two media with different refractive indices of n₁ and n₂, the amplitude reflectance r is

$$r = \frac{{n}_{1} - {n}_{2}}{{n}_{1} + {n}_{2}} , \qquad ( 15 )$$

[see the reference: J. M. Bennett, “Polarization,” Chapter 5, Handbook of Optics, Vol. I, 2ed, Edited by M. Bass, and et al, McGRAW-Hill, New York, 1995, p.5.7], and the light intensity reflectance R=r². We get the light intensity reflectance R=0.0011 for this interface, Also according to the intensity enhancement factor γ and the intensity attenuation factor ε shown in the Table 1, we get the signal intensity change factor ξ and the imaging sensitivity enhancement factor α values with different processing or imaging distances of 2 cm, 5 cm, 10 cm, 15 cm and 20 cm in the human body as the follows in Table 1.

The absorption coefficient and scattering coefficient of the seawater vary a lot according to the different situations, here taking the clear seawater as the example. The absorption coefficient μ_{wa} of clear seawater is 0.0196 m⁻¹, and its scattering coefficient μ_{ws} is 0.0212 m⁻¹ [see above reference written by C. D. Mobley]. The refractive index of water at the wavelength of 550 nm is 1.336. Assuming the refractive index of the object is 1.6 (note that the optical glass refractive index range is 1.5 to 2.0), then an assumed light intensity reflectance of R=0.00809 for object in clear seawater is obtained. According to the intensity enhancement factor γ of the peak light intensity and the intensity attenuation factor ε of the remain light intensity in the Table 2, we get the signal intensity change factor ξ and the imaging sensitivity enhancement factor α values with different processing or imaging distances of 200 m, 500 m, 1000 m, 1500 m and 2000 m in the clear seawater shown in Table 2 (also see attached appendix for the detailed descriptions about the calculations).

From Table 1 and 2, we can see that the signal composite light intensity maximum is much higher than the signal intensity of the normal imaging. For example, when D′=5 cm for medical processing or imaging, or D′=500 m for underwater processing or imaging, the intensity enhancement factor is more than 1.1×10³ when N>10³. It means that when N>10³, and the total spectra width of N beams is wide enough, the peak intensity of the signal light pulse can be higher than NI₀.Note that NI₀ is the average value of the total intensity of the N beams (if the N beams are incoherent light beams). Of course, the extreme high pulse peak intensity is always with the extreme narrow pulse duration usually, and so the energy of each pulse may be very low.

However, as long as the signal to noise ratio is high, the required signal energy can be got by receiving repeated signal pulses. It can be seen that the value ξ is still high even when D′=20 cm for medical processing or imaging, or D′=2000 m for underwater processing or imaging. Therefore, there is good potential to get the signal intensity enhancement factor of near 2000 dB, whose corresponding processing or imaging depth and distance are 20 cm in the human body and 2000 m in the clear seawater. Considering the approximations are made in the calculations, the signal intensity enhancement factor of 600 dB is taken for representing the apparatus performances, whose corresponding processing or imaging depth is 5 cm in the human body, and 1000 m in the clear seawater.

TABLE 2
Signal Intensity Enhancement at Different Imaging Depth in Seawater by Multi-beam Interference

| N | Γ | ε | Factor | D′ = 200 m | D′ = 500 m | D′ = 1000 m | D′ = 1500 m | D′ = 2000 m |
|---|---|---|---|---|---|---|---|---|
| 1.00E+01 | 1.00E+02 | 5.05E−14 | ξ | 1.10E−01 | 1.10E−01 | 1.10E−01 | 1.10E−01 | 1.10E−01 |
| 1.00E+02 | 1.00E+04 | 6.11E−10 | ξ | 1.10E+01 | 1.10E+01 | 1.10E+01 | 1.10E+01 | 1.10E+01 |
| 1.00E+03 | 9.97E+05 | 6.21E−06 | ξ | 1.10E+03 | 1.10E+03 | 1.09E+03 | 1.09E+03 | 1.09E+03 |
| 1.00E+04 | 7.08E+07 | 2.80E−06 | ξ | 7.79E+04 | 7.79E+04 | 7.78E+04 | 7.78E+04 | 7.78E+04 |
| 1.00E+05 | 7.08E+09 | 1.13E−05 | ξ | 7.78E+06 | 7.78E+06 | 7.76E+06 | 7.75E+06 | 7.74E+06 |
| 1.00E+06 | 7.08E+11 | 9.35E−06 | ξ | 7.79E+08 | 7.78E+08 | 7.77E+08 | 7.76E+08 | 7.75E+08 |
| 1.00E+07 | 7.08E+13 | 5.31E−06 | ξ | 7.79E+10 | 7.78E+10 | 7.78E+10 | 7.77E+10 | 7.77E+10 |
| 1.00E+01 | 1.00E+02 | 5.05E−14 | α (dB) | 2.63E+02 | 6.27E+02 | 1.23E+03 | 1.84E+03 | 2.45E+03 |
| 1.00E+02 | 1.00E+04 | 6.11E−10 | α (dB) | 2.83E+02 | 6.47E+02 | 1.25E+03 | 1.86E+03 | 2.47E+03 |
| 1.00E+03 | 9.97E+05 | 6.21E−06 | α (dB) | 3.03E+02 | 6.67E+02 | 1.27E+03 | 1.88E+03 | 2.49E+03 |
| 1.00E+04 | 7.08E+07 | 2.80E−06 | α (dB) | 3.21E+02 | 6.85E+02 | 1.29E+03 | 1.90E+03 | 2.51E+03 |
| 1.00E+05 | 7.08E+09 | 1.13E−05 | α (dB) | 3.41E+02 | 7.05E+02 | 1.31E+03 | 1.92E+03 | 2.53E+03 |
| 1.00E+06 | 7.08E+11 | 9.35E−06 | α (dB) | 3.61E+02 | 7.25E+02 | 1.33E+03 | 1.94E+03 | 2.55E+03 |
| 1.00E+07 | 7.08E+13 | 5.31E−06 | α (dB) | 3.81E+02 | 7.45E+02 | 1.35E+03 | 1.96E+03 | 2.57E+03 |

As shown in Table 1 and Table 2, the enhancement of the imaging sensitivity of the said apparatuses is very large. The said apparatuses have such great performance is not strange, because it is created by multiple beam interference. In the past, multiple beam interference has demonstrated its astonishing abilities, such as to create extremely short light pulse of dozens of attoseconds (1 attosecond=10⁻¹⁸ s) and extremely strong light power of several terawatts (1 terawatt=10¹² watts). They are the fastest-ever and strongest-ever man-made events until now. In the future, the multiple beam interference will surely make more technical contributions.

At last, we give the practical method for adjusting the additional distance to get accurate expected imaging and laser processing distance in the turbid media or human body. In a turbid medium or human body, there are various compositions with different refractive indices, so it is difficult to find the accurate value of the additional distance determined by all refractive indices of the compositions in the turbid medium or human body. However, there is a practical way to overcome this difficulty. Because when the angular phase differences between any frequency adjacent beams become zero, the composite light intensity maximum emerges certainly. Therefore, somewhat like to search for a music station by tuning the frequency of a radio, no matter what the accurate distance of the object or the targeted tissue position is, one just needs observing into the turbid medium or human body and adjusting the distance adjuster at the same time. When the searched object or targeted tissue appears in the visual field (by camera) and becomes clear, the accurate expected processing or imaging distance is achieved.

The acoustic wave has good ability to permeate dense liquid and solid materials when the wave frequency is not high. It is used widely for detecting the information in dense materials, such as the injury in bulk solid materials. Therefore, to increase acoustic imaging and processing distance in dense materials is very useful.

Because optical waves and acoustic waves all are oscillation waves, such waves all cosinusoidally vary, that is, these waves all have cosine or sine forms. Therefore, multiple beam (wave) interference can certainly happen to them, and result in the similar physical results, that is, using multiple wave interference to reduce the composite wave intensity in the propagation path, and increase composite wave intensity at expected positions. Therefore, the apparatus of acoustic processing or imaging/detection in dense materials, that is, in turbid media can be designed based on the principle of this invented method.

The apparatus of acoustic processing with or without image-guild for sound wave absorption or/and scattering materials designed based on the method of laser beam processing for light absorption and scattering materials comprising: a sound wave generator generating N sound waves with different frequencies and the same or not same frequency intervals, the same or approximately the same amplitudes, and the zero initial phases at a certain moment; a mirrored negative dispersion generation device for sound waves; an processing or/and imaging distance adjuster to adjust the expected processing or/and imaging distance in the material, a means to move the processing or/and imaging area in the material in three dimensions.

The said mirrored negative dispersion generation device for sound waves generates the acoustic path difference compensations for all pairs of two frequency adjacent sound waves of the said N sound waves for the acoustic path differences produced in the material contains the object for all pairs of two frequency adjacent sound waves of the said N sound waves.

The said sound wave generator may be a mode-locked acoustic laser. The said imaging or detection distance adjustor consists of the components similar to those in the above described medical and underwater imaging apparatuses, but can make acoustic wave traveling distance change. The said means to move imaging or detecting area in the turbid media in three dimensions is using the reflector(s) for acoustic waves. Because the acoustic imaging or detection apparatus can be designed based on the same principle of this invented method and existing knowledge, no more description is given here.

Similarly, based on the principle of this invented method, a variety of existing technologies can combined to create a variety of new functions. Since these works may be done by using the existing knowledge, no more descriptions are given here.

I claim:

1. A method of laser beam processing for light absorption and scattering materials, the method comprising: using a negative dispersion generation device to broaden the full width of half maximum of a short light pulse; then making the broadened light pulse enter the material containing the object for processing; also utilizing positive dispersion of the material containing the object to compress the broadened light pulse in the propagation path, and to create a short light pulse again which forms an inner light layer to illuminate the object in the material; if needed, making the short signal light pulse reflected from the object return along the incident path reversely; during the return path, the full width of half maximum of the short signal light pulse is broadened by positive dispersion of the material again; then, the broadened signal light pulse is compressed by the negative dispersion generation device; and the broadened signal light pulse becomes short signal light pulse again and is received by imaging receiver placed at the observing position for image-guided processing; and at last increasing the power of the illumination short light pulse to a required value, the object can be processed.

2. The method of laser beam processing for light absorption and scattering materials of claim 1, wherein the frequency range of said short light pulse is in visible region, or/and in infrared, or/and in ultraviolet, or/and in X-ray region(s).

3. The method of laser beam processing for light absorption and scattering materials of claim 1, wherein said image receiving position is designed by making the absolute value of the dispersion generated by the negative dispersion generation device be equal to the absolute value of the dispersion generated by the material within the path reaching the object, but with the opposite sign, and making two optical path distances of the output port of the said short light pulse source and the image receiving position to the object in the material be equal.

4. The method of laser beam processing for light absorption and scattering materials of claim 1, wherein said inner light layer is plane, or cylindrical, or spherical layer located in the material, the thickness of the layer is much thinner than the processing or imaging distance in the material, or this layer is focused to be point or line located in the material.

5. The method of laser beam processing for light absorption and scattering materials of claim 1, wherein said short light pulse may be used to process or/and image the object repeatedly in the material.

6. The method of laser beam processing for light absorption and scattering materials of claim 1, wherein said increasing the power of the illumination short light pulse to a required value is to make the peak intensity of the formed illumination short light pulse in the material be high enough for processing the object, and to make the light intensity of the illumination light pulse in the propagation path be low enough for not damaging the material by light pulse broadening.

7. A apparatus of laser beam processing for light absorption and scattering materials designed based on the method of claim 1, the apparatus comprising: laser generating short light pulse which contains N polarized light beams with different frequencies, the same or approximately the same polarization states, and zero initial phases at a certain moment; the angular frequency intervals Δω of these N beams are equal or not equal but are equal usually; an optical adjusting means to make the amplitudes of the N polarized light beams become the same or approximately the same; a mirrored negative dispersion generation device; an processing or imaging distance adjuster to adjust the processing or imaging distance in the material containing the object; a means to move the formed inner light layer, or light point, or light line in the material in three dimensions.

8. The apparatus of laser beam processing for light absorption and scattering materials of claim 7, wherein said light absorption and scattering materials include human body, animal body, seawater, river water, lake water, pond water, fog, smog, snow, ice, cloud, atmosphere, and any gaseous, liquid or solid materials which have light absorption or/and scattering, especially have strong light absorption or/and scattering.

9. The apparatus of laser beam processing for light absorption and scattering materials of claim 7, wherein said laser beam processing includes medical laser beam treatments, medical laser beam surgery, light communications in atmosphere or water, various light energy delivery in bulk gas, bulk liquid and bulk solid materials for heating, denaturing, ablating, etching, welding, drilling, vaporizing, hitting, cutting, destroying, and so on.

10. The apparatus of laser beam processing for light absorption and scattering materials of claim 7, wherein said laser is mode-locked laser.

11. The apparatus of laser beam processing for light absorption and scattering materials of claim 7, in the wherein said apparatus, the optical elements including the prisms, triangular components, lenses, beam splitters, and so on, all are made of the same material as the material for processing or/and imaging, or all are made of the material which has the same or very approximate same dispersion property as that of the material for processing or/and imaging.

12. The apparatus of laser beam processing for light absorption and scattering materials of claim 7, wherein the number N of said N polarized light beams is from 3 to 10¹² or more, the angular frequency intervals Δω of any two frequency adjacent beams of these N beams are equal or not equal but are equal usually.

13. The apparatus of laser beam processing for light absorption and scattering materials of claim 7, wherein said N polarized light beams are plane polarized, or elliptically polarized, or circularly polarized light beams, wherein said polarization states include polarization directions of the plane polarized light beams, ellipticities of the elliptically polarized light beams.

14. The apparatus of laser beam processing for light absorption and scattering materials of claim 7, wherein said N light beams are plane, or cylindrical, or spherical light beams.

15. The apparatus of laser beam processing for light absorption and scattering materials of claim 7, wherein said mirrored negative dispersion generation device consists chiefly of the prisms and lenses, the output surface of the prism generating the negative dispersion is shaped by computer-controlled high precision grounding and polishing to satisfy the requirements of optical path difference compensations for all pairs of two frequency adjacent beams of the said N beams, and the retroreflective micro-mirror layer is used to make the output surface of the prism generating the negative dispersion become retroreflective surface for reflecting the said N beams with different incident angels reversely.

16. The apparatus of laser beam processing for light absorption and scattering materials of claim 7, wherein said processing or/and imaging distance adjuster consists of two triangular components, which move in opposite directions to adjust the processing or/and imaging distance in the material by changing an additional distance outside the material and offset the extra dispersions caused by component triangular shapes.

17. The apparatus of laser beam processing for light absorption and scattering materials of claim 7, wherein said means to move the said inner light layer, or said point, or said line in the material in three dimensions is by moving reflective mirror(s) to make N polarized light beams scan in two dimensional plane, and adjusting the imaging and processing distance in third dimension.

18. The apparatus of laser beam processing for light absorption and scattering materials of claim 7, wherein said an optical adjusting means to make the amplitudes of N polarized light beams become the same or approximately the same is using dye to make dispersion compensation to laser cavity gain.

19. A apparatus of acoustic processing with or without image guide for sound wave absorption or/and scattering materials designed based on the method of laser beam processing for light absorption and scattering materials of claim 1, the apparatus comprising: a sound wave generator generating N sound waves with different frequencies and the same or not same frequency intervals, the same or approximately the same amplitudes, and the zero initial phases at a certain moment; a mirrored negative dispersion generation device for sound waves; an processing or/and imaging distance adjuster to adjust the expected processing or/and imaging distance in the material, a means to move the processing or/and imaging area in the material in three dimensions.

20. The apparatus of acoustic processing with or without image guide for sound wave absorption or/and scattering materials of claim 19, wherein said mirrored negative dispersion generation device for sound waves generates the acoustic path difference compensations for all pairs of two frequency adjacent sound waves of the said N sound waves for the acoustic path differences produced in the material contains the object for all pairs of two frequency adjacent sound waves of the said N sound waves.
